(19) United States
(12) Patent Application Publication
(10) Pub. No.: US 2026/0137963 A1
(43) Pub. Date: May 21, 2026

(54) METHODS AND SYSTEMS OF IMPROVING MEDICAL CONDITIONS VIA ULTRASOUND NEUROMODULATION OF THE BRAIN

(71) Applicant: West Virginia University Board of Governors on behalf of West Virginia University, Morgantown, WV (US)

(72) Inventors: Ali Rezai, Morgantown, WV (US); Victor Finomore, Morgantown, WV (US)

(21) Appl. No.: 19/369,485

(22) Filed: Oct. 27, 2025

Related U.S. Application Data
(60) Provisional application No. 63/712,569, filed on Oct. 28, 2024.

(51) Int. Cl. A61N 7/00

(52) U.S. Cl. CPC A61N 7/00; A61N 2007/0026

(57) ABSTRACT

Methods and systems for improving medical conditions by delivering a focused ultrasound signal to target sites of the brain. The focused ultrasound signal can have a mechanical index of between about 1.0 to about 8.0 and/or an acoustic pressure of between about 1.0 MPa to about 3.0 MPa. The medical condition can comprise addiction (including binge eating), anxiety disorders and anxiety associated disorders, tinnitus, neurodegenerative disorders, neuropsychiatric disorders, cognitive disorders, neurodevelopmental disorders, post-traumatic stress disorder, stroke recovery, and combinations thereof.

### CROSS REFERENCE TO RELATED APPLICATIONS

The present application claims priority to U.S. Application No. 63/712,569 filed on Oct. 28, 2024 and is incorporated by reference herewith in its entirety.

### TECHNICAL FIELD

The present technology is directed to methods and systems for improving medical conditions by delivering a focused ultrasound signal (FUS) to a neural target site of brain.

### BACKGROUND

Neuromodulation is a series of techniques that can alter nerve activity in the body to normalize and improve nervous tissue function. It can involve applying stimuli, such as electrical stimulation or chemical agents, to specific neurological sites. Neuromodulation can help decrease pain, reduce tremor and anxiety and to increase mobility by changing the way nerves carry information to and from the brain.

Neuromodulation can be used to treat a wide range of conditions involving nerves, muscles, and brain function. For example, transcutaneous electrical nerve stimulation (TENS) units and implanted spinal cord stimulators are based on the concept of neuromodulation and can provide substantial relief for many patients. Neuromodulation can also be used to treat treatment-resistant depression.

### SUMMARY

In an aspect, a method of improving a medical condition in a patient in need thereof is provided. The method can comprise delivering a focused ultrasound neuromodulation signal to a neural target site of the patient's brain. The ultrasound signal can have a mechanical index of between about 1.0 to about 8.0 and/or an acoustic pressure of between about 1.0 MPa to about 3.0 MPa, the medical condition comprising addiction (including binge eating), anxiety disorders and anxiety associated disorders, tinnitus, neurodevelopmental disorders, neurodegenerative disorders, post-traumatic stress disorder, stroke recovery, or combinations thereof.

In another aspect, a computer program is provided comprising instructions, which, when the program is executed by a processor for controlling an ultrasound pulse generator causes the step of delivering a focused ultrasound neuromodulation signal to a neural site of the brain, the ultrasound signal having a mechanical index of between about 1.0 to about 8.0 and/or an acoustic pressure of between about 1.0 MPa to about 3.0 MPa and to a site of the brain sufficient to improve addiction (including binge eating), anxiety disorders and anxiety associated disorders, tinnitus, neurodegenerative disorders, neuropsychiatric disorders, cognitive disorders, neurodevelopmental disorders, post-traumatic stress disorder, stroke recovery, and combinations thereof.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates a system for providing focused ultrasound treatment for treatment or diagnosis of a disorder to a specific region of interest;

FIG. 2 represents a method for screening a patient for a specific disorder;

FIG. 3 illustrates a method for using cues to generate features used to screen patients for a disorder;

FIG. 4 illustrates one method for monitoring a patient to predict or detect an onset of symptoms associated with a disorder for a patient;

FIG. 5 illustrates another example of a method for evaluating a patient for an onset of symptoms requiring treatment via focused ultrasound treatment;

FIG. 6 illustrates another method for targeting focused ultrasound treatment in a for treatment of a disorder using acute feedback; and

FIG. 7 illustrates a method for selecting a dose for focused ultrasound treatment.

### DETAILED DESCRIPTION

The present disclosure relates to methods and systems for improving medical conditions via ultrasound neuromodulation of the brain. In an aspect, a method of improving a medical condition in a patient in need thereof can comprise delivering a focused ultrasound neuromodulation signal to a neural target site of the patient's brain, the ultrasound signal having an mechanical index of between about 1.0 and about 8.0 and/or an acoustic pressure of between about 1.0 MPa and about 3.0 MPa. The focused ultrasound neuromodulation signal can be ablative or non-ablative. The medical condition can comprise addiction (including binge eating), anxiety disorders and anxiety associated disorders, tinnitus, neurodegenerative disorders, neuropsychiatric disorders, cognitive disorders, neurodevelopmental disorders, post-traumatic stress disorder, stroke recovery, and combinations thereof. In certain aspects, a clinical or feedback parameter (as disclosed in more detail below) is used to determine whether to apply and/or adjust delivery ultrasound therapy.

In another aspect, a computer program is provided that comprises instructions, which, when the program is executed by a processor for controlling an ultrasound pulse generator causes the step of delivering a focused ultrasound neuromodulation signal to a neural site of the brain. The ultrasound signal can have a mechanical index of between about 1.0 and about 8.0 and/or an acoustic pressure of between about 1 MPa and about 3.0 MPa. The ultrasound signal is delivered to a site of the brain sufficient to improve addiction (including binge eating), anxiety disorders and anxiety associated disorders, tinnitus, neurodegenerative disorders, neuropsychiatric disorders, cognitive disorders, neurodevelopmental disorders, post-traumatic stress disorder, stroke recovery, and combinations thereof. In certain aspects, the processor can apply or adjust application of the ultrasound signal based in clinical/feedback parameters disclosed in more detail below.

As used herein with respect to a described element, the terms “a,” “an,” and “the” include at least one or more of the described element(s) including combinations thereof unless otherwise indicated. Further, the term “or” includes “and” and combinations thereof unless otherwise indicated. The term “and” refers to combinations thereof unless otherwise indicated. By “substantially,” “approximately, or “about” is meant that the shape, size, configuration or value of the described element need not have the mathematically exact described shape, size, configuration or value of the described element but can have a shape, size, configuration or value that is recognizable by one skilled in the art as generally or approximately having the described shape, size, configuration, or value of the described element. As such “substantially,” “approximately,” or “about” refers to the complete or nearly complete extent of a characteristic, property, state, structure or value. The exact allowable degree of deviation from the characteristic, property, state, structure, or value will be so as to have the same overall result as if the absolute characteristic, property, state, structure, or value were obtained. The terms “first,” “second,” etc. are used to distinguish one element from another and not used in a quantitative sense unless indicated otherwise. Thus, a “first” element described below could also be termed a “second” element. A “patient” as used herein is a mammal such as, for example, a human being, dog, cat, horse, pig, sheep, cow, or other domesticated animal.

As used herein, a “clinical parameter” is any value representing a patient that is relevant to the patients' risk of a disorder or a progression of a disorder. Clinical parameters can include values measured in a clinical environment, values measured outside of a clinical environment by one or more wearable or portable devices, or values retrieved from an electronic health records (EHR) interface and/or other available databases. It will be appreciated that the clinical parameter can also be referred to herein as “feedback parameter” including, for example, a physiological, cognitive, behavioral, psychosocial or anatomical parameter associated with the patient.

As used herein, a “predictive model” is a mathematical model or machine learning model that either predicts a future state of a parameter or estimates a current state of a parameter that cannot be directly measured. A predictive model is trained on training samples, each training sample comprising a set of values used for predicting the parameter in the predictive model and a known value for the parameter. It will be appreciated that a predictive model can be implemented as machine executable instructions.

As used herein, a “categorical value” is a value that can be represented by one of a number of discrete possibilities, which may or may not have a meaningful ordinal ranking. A “continuous value,” as used herein, is a value that can take on any of a number of numerical values within a range. It will be appreciated that, in a practical application, values are expressed in a finite number of significant digits, and thus a “continuous value” can be limited to a number of discrete values within the range.

As used herein, data is provided from a first system to a second system when it is either provided directly from the first system to the second system, for example, via a local bus connection, or stored in a local or remote non-transitory memory by the first system for later retrieval by the second system. Accordingly in some implementations, the first system and the second system can be located remotely and in communication only through an intermediate medium connected to at least one of the systems by a network connection.

“Registration” of two or more images includes any process that assigns relative locations between pixels or multi-pixel features across two or more images. This assignation can be represented, for example, via an explicit transformation model between two or more images or via feature matching techniques that identify common structural features across two images.

An “average,” as used herein, can be any measure of central tendency, including but not limited to, an arithmetic mean, a geometric mean, a median, and a mode. It will be appreciated that, where a mean for a set of values is used as the average, the mean can be taken from a subset of the set of values to eliminate outliers within the set of values. For example, values between the fifth and the ninety-fifth percentile can be used to generate the mean.

An “intensity profile,” as used herein, represents a spatial variation in the intensity of localized energy provided to a target. An intensity profile can include a variance between two sides of a region provided with the localized energy or a more complex spatial variation of the energy.

### Focused Ultrasound

Ultrasound can be defined as an acoustic wave above 20 kHz, beyond the frequency range of human hearing. In a typical neuromodulation procedure, a pulse generator emits an electrical waveform, which is amplified and carried to an ultrasound transducer housing a piezoelectrical element coupled to its active face. An ultrasound transducer and ultrasound device are used interchangeably herein. The amplified electrical signal excites the piezoelectric element, which mechanically oscillates the active face of the transducer.

### FUS Parameters

Table I provides examples of FUS/sonication parameters that can be measured.

TABLE I
Sonication parameters.

| | Abbrev. | |
|---|---|---|
| Parameter | (Unit) | Description |
| Ultrasound | f₀ (kHz or | The frequency of ultrasonic pressure |
| operating | MHz) | waves; the carrier frequency of the |
| frequency | | ultrasound signal |
| Pulse duration | PD (ms) | The shortest continuous period of |
| | | sonication. When applying continuous |
| | | TUS, pulse duration is equal to the |
| | | pulse train duration; the duration of a |
| | | pulse in a series of ultrasound pulses |
| Pulse repetition | PRF (Hz) | The frequency of pulse delivery within |
| frequency/rate | | the pulse train duration; multiplicative |
| | | inverse of the pulse repetition interval |
| Duty cycle | DC (%) | The percentage of time sonication is |
| | | delivered during the pulse repetition |
| | | interval. The duty cycle is only defined |
| | | for rectangular pulses. (or pulse train |
| | | duration) |
| Spatial | pₛₚ | The position at which the ultrasound |
| peak/spatial peak | | signal has a highest amplitude. The |
| pressure | | spatial peak occurs within a focal spot |
| | | of the ultrasound transducer; the |
| | | amplitude of the pressure during the |
| | | steady part of the pulse at the location |
| | | of the spatial peak. |
| Spatial peak pulse | I_{SPPA} | The intensity of the pulse at the spatial |
| average intensity | (W/cm²) | peak (focal peak) time averaged over |
| | | the pulse duration |
| Spatial peak | I_{SPTA} | The intensity of the ultrasonic stimulus |
| temporal average | (mW/cm²) | at the spatial peak time averaged over |
| intensity | | a defined period of time (i.e., across the |
| | | stimulus duration). The temporal |
| | | window must be defined. |
| Mechanical Index | MI | A unitless index for assessing the |
| | | likelihood of cavitational bioeffects. |
| | | Where p_{r.3} is the pressure derated using |
| | | αα = 0.3 dB cm⁻¹ MHz⁻¹ |
| Acoustic Pressure | Megapascal | Amount of force applied per unit area |
| | (MPa) | and is typically thought of as a local |
| | | pressure deviation relative to the |
| | | ambient surround pressure |
| Power | Watts | The power of the electrical waveform |
| | | emitted by ultrasound pulse generator |
| | | which is amplified and carried to one or |
| | | more ultrasound transducers |

Z = Acoustic Impedance

In further detail, to characterize the intensity of a pulsing protocol, a needle hydrophone can be used to measure the instantaneous pressure (Pᵢ) in Megapascals applied by the transducer in an acoustic medium. The instantaneous intensity (Iᵢ) is proportional to the square of the instantaneous pressure, and inversely related to the density (ρ) and speed of sound (c) in the propagating medium. The pulse intensity integral (PII) is then derived by integrating the instantaneous intensity over the duration of the pulse. From the PII, two measures of acoustic exposure can be derived: the spatial-peak temporal average (I_{SPTA}) and the spatial-peak pulse average (I_{SPPA}). I_{SPTA} measures the average intensity during the entire sonication and scales in proportion to sonication duration. Conversely, I_{SPPA} represents the average intensity over a single pulse, providing an estimate of short-term mechanical bioeffects. Another parameter is acoustic pressure, which is the amount of force applied per unit area and is typically thought of as a local pressure deviation relative to the ambient surround pressure. It is commonly represented with the symbol p and is given in Megapascal (MPa) units. Another parameter that can be measured is the mechanical index (MI), a unitless measure which is equal to peak negative pressure divided by the square of the fundamental frequency. The MI can estimate the risk of potentially destructive biomechanical effects on tissues, such as inertial cavitation. Frequency can also be measured, which is the number of wave cycles per second and is determined by the rate at which the ultrasound source oscillates and influences many aspects of tissue interaction. It is commonly represented with the symbol f and is given in Megahertz (MHz) units.

### FUS Parameter Values

The focused ultrasound signal can be generated with suitable stimulation parameters

In an example, focused ultrasound is provided with a mechanical index between 0.1 and 8.0. In a further example, focused ultrasound is provided with a mechanical index between one and five. In another example, the mechanical index is between 1.5 to 5. In certain aspects, the mechanical index is 4.2 at a frequency of 220 kHz and a mechanical index of 2.6 at a frequency of 500 kHzIn certain aspects, the mechanical index is about 2.7, about 4.2 or less than about 0.8.

In one implementation, focused ultrasound treatment is provided with a pulse duration between 3 msec and 300 msec. In another implementation, the focused ultrasound treatment is provided with a pulse duration of 100 ms. In another implementations, the focused ultrasound treatment is provided at pulse duration of 150 ms.

In one example, focused ultrasound treatment is provided with an acoustic pressure between 1.0 MPa and 3.0 MPa. In another example, focused ultrasound is delivered with an acoustic pressure of 2.5 MPa. In another example, focused ultrasound is delivered with an acoustic pressure of 2 MPa. In another example, focused ultrasound is delivered with an acoustic pressure between about 1.84 and 1.9 MPa. In certain aspects, the acoustic pressure is greater than zero but less than about 2 MPa, such as about 0.55 MPa or about 1.9 MPa.

In one implementation, a session of focused ultrasound treatment lasts between three minutes and seven minutes. In another implementation, a session of focused ultrasound treatment lasts between five minutes and twenty minutes. In a further implementation, a session of focused ultrasound treatment lasts between three minutes and ten minutes. In a further implementation, a session of focused ultrasound treatment lasts between five minutes and twenty minutes. In a further implementation, a session of focused ultrasound treatment lasts between ten minutes and twenty minutes. In a further implementation, a session of focused ultrasound treatment lasts between ten minutes and thirty minutes. In a further implementation, a session of focused ultrasound treatment lasts between ten minutes and sixty minutes.

In one implementation, focused ultrasound treatment is provided with a carrier frequency between about 0.22 MHz and 3 MHz. In another implementation, focused ultrasound treatment is provided with a carrier frequency of about 220 kHz. In another implementation the focused ultrasound is delivered at about 500 kHz. In certain aspects, the carrier frequency is between about 0.22 and about 0.65 MHz. In certain aspects, the carrier frequency is about 0.6 MHz.

In one implementation, focused ultrasound treatment is provided with a spatial-peak temporal average intensity between 0.1 W/cm² and 8.5 W/cm². In another implementation, focused ultrasound treatment is provided with a spatial peak average intensity is between 3 W/cm² and 8.5 W/cm². In a further implementation the focused ultrasound treatment is provided with a spatial-peak temporal average intensity between about 1.33 W/cm² and about 5.64 W/cm².

In certain aspects, the I_{SPPA} can greater than zero and up to about 110 W/cm². In certain aspects, the I_{SPTA} can be greater than zero and up to about 8 W/cm². In certain aspects, the duty cycle can be greater than 0% and less about than 30%. In certain aspects, the duty cycle can be about 6.6%. In certain aspects, the duty cycle can be about 3.3%. In certain aspects, the duty cycle can be about 5%. In certain aspects, the pulse repetition rate can be between about 0.33 Hz and about 100 Hz. In certain aspects, the pulse repetition rate is about 0.33 Hz. In certain aspects, the pulse duration is between about 3 ms and about 300 ms. In certain aspects, the pulse duration is about 100 ms. In certain aspects, the acoustic pressure is between about 0.1 MPa and about 2.5 MPa. In certain aspects, the acoustic pressure is about 2 MPa. In certain aspects, the mechanical index is between about 0.1 and about 5. In certain aspects, the mechanical index is 4. In certain aspects, a session of focused ultrasound treatment lasts greater than zero minutes and up to about 30 minutes.

### Medical Conditions

The present disclosure provides methods of improving a medical condition in a patient in need thereof. The methods include delivering a focused ultrasound signal to a neural target site of the patient's brain. The medical condition can comprise, for example, addiction (including binge eating), anxiety disorders and anxiety associated disorders, tinnitus neurodegenerative disorders, neuropsychiatric disorders, cognitive disorders, neurodevelopmental disorders, post-traumatic stress disorder, stroke recovery, and combinations thereof.

Also provided herein are computer programs comprising instructions, which, when the program is executed by a processor for controlling an ultrasound device or transducer causes the steps of delivering a focused ultrasound signal to a neural site of the brain. The ultrasound signal can delivered to a neural target site of the brain sufficient to improve, for example, addiction (including binge eating), anxiety disorders and anxiety associated disorders, tinnitus neurodegenerative disorders, neuropsychiatric disorders, cognitive disorders, neurodevelopmental disorders, post-traumatic stress disorder, stroke recovery, and combinations thereof.

### Systems

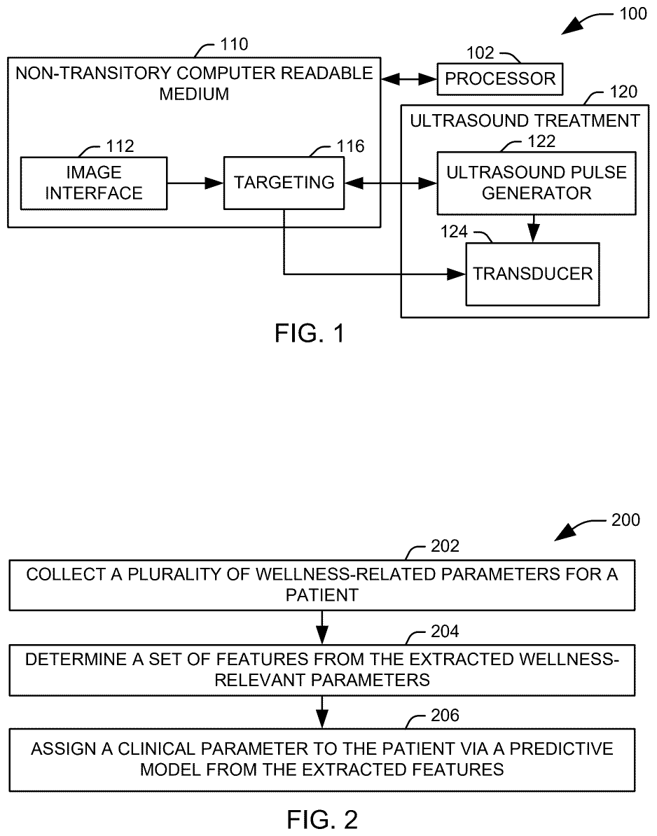

FIG. 1 illustrates a system 100 for providing focused ultrasound treatment for treatment or diagnosis of a disorder to a specific region of interest. The system 100 includes a processor 102 and a non-transitory computer readable medium 110 that stores executable instructions for targeting focused ultrasound treatment for treatment or diagnosis of disorders. The executable instructions include an imaging interface 112 that receives an image representing a region of interest for a patient from one or more associated imaging systems (not shown). The imaging interface 112 can include appropriate software components for communicating with an imaging system (not shown) or repository of stored images (not shown) over a network via a network interface (not shown) or via a bus connection. The image can be provided to a targeting component 116 that selects a location and intensity profile for the focused ultrasound treatment based on the image, the location, the type of tissue, and one or more feedback parameters of the patient, such as those listed in Tables II through VIII below.

In one example, the targeting component 116 can operate in conjunction with a focused ultrasound treatment system 120, comprising an ultrasound pulse generator 122 and at least one transducer 124 driven by the ultrasound pulse generator. Specifically, the targeting component can instruct the ultrasound pulse generator 122 to deliver a focused ultrasound neuromodulation signal to a neural site of the brain, with the ultrasound signal having a mechanical index of between about 1 and about 4 and/or an acoustic pressure of between about 1 MPa and about 2.5 MPa to a site of the brain sufficient to improve addiction (including binge eating), anxiety disorders and anxiety associated disorders, tinnitus, neurodegenerative disorders, neuropsychiatric disorders, cognitive disorders, neurodevelopmental disorders, post-traumatic stress disorder, stroke recovery, and combinations thereof.

### Methods of Screening

FIG. 2 represents a method 200 for screening a patient for a specific disorder. At 202, a plurality of parameters is collected for the patient. Parameters can be retrieved from an electronic health records (EHR) interface and/or other available databases, including, for example, employment information (e.g., title, department, shift), age, sex, home zip code, genomic data, nutritional information, medication intake, household information (e.g., type of home, number and age of residents), social and psychosocial data, consumer spending and profiles, financial data, food safety information, the presence or absence of physical abuse, and relevant medical history. Parameters can also be measured via diagnostic systems, imaging systems, wearable sensors, cognitive tests, questionnaires, and other means. Relevant parameters can include at least physiological, cognitive, motor/musculoskeletal, sensory, sleep, biomarkers and behavioral parameters.

In aspects where a patient's physiological parameter is measured, the physiological parameter can be a response of the patient's brain to cue exposure and multiple physiological parameters can be measured during any given assessment session. The physiological parameters can be measured via a wearable device such as a ring, watch, or belt or via a smart phone or tablet, for example, in a naturalistic non-clinical setting such as when the patient is at home, work or other non-clinical setting. Exemplary physiological parameters include heart rate, heart rate variability, perspiration, salivation, blood pressure, pupil size, brain activity, electrodermal activity, body temperature, and blood oxygen saturation level. Table II provides non-limiting examples of physiological parameters that can be measured and exemplary tests to measure the physiological parameters.

### Clinical/Feedback Parameters

TABLE II

| Physiological | Exemplary Devices and Methods to Measure |
|---|---|
| Parameter | Physiological Parameters |
| Brain Activity | Electroencephalogram, Magnetic Resonance Imaging |
| & Brain | (MRI), including functional Magnetic Resonance |
| Structure | Imaging (fMRI), positron emission tomography (PET), |
| | single-photon emission computer tomography |
| | (SPECT), magnetoencephalography (MEG), near- |
| | infrared spectroscopy (NIRS), functional near-infrared |
| | spectroscopy (f-NIRS), trbraincranial doppler |
| | ultrasound (TCD), and other brain imaging modalities |
| | looking at electrical, blood flow, neurotrbrainmitter, |
| | and metabolic MRI, taken either during a cognitive |
| | task or while the patient is at rest |
| Heart rate | Electrocardiogram and Photoplethysmogram |
| Heart rate | Electrocardiogram, Photoplethysmogram |
| variability | |
| Eye tracking | Pupillometry, including tracking saccades, fixations, |
| | and pupil size (e.g., dilation) |
| Perspiration | Perspiration sensor |
| Blood pressure | Sphygmomanometer |
| Body temperature | Thermometer, infrared thermography |
| Blood oxygen | Pulse oximeter/accelerometer |
| saturation and | |
| respiratory rate | |
| Skin conductivity | Electrodermal activity |
| Facial emotions | Camera or EMG based sensors for emotion and |
| | wellness |
| Sympathetic and | Derived from the above measurements |
| parasympathetic | |
| tone | |

The physiological parameters can be measured in clinical settings with appropriate devices or in non-clinical settings via wearable, implantable, or portable devices. Some information can also be determined from self-reporting by the user via applications in a mobile device or via interaction with applications on the mobile device. For example, a smart watch, ring, or patch can be used to measure the user's heart rate, heart rate variability, body temperature, blood oxygen saturation, movement, and sleep. In a non-clinical setting, these values can also be subject to a diurnal analysis to estimate variability. Eye tracking can be performed, for example, using a camera on a mobile device and specialized software.

Table III provides non-limiting examples of cognitive parameters that are gamified and that can be measured and exemplary methods and tests/tasks to measure such cognitive parameters. The cognitive parameters can be assessed by a battery of cognitive tests that measure, for example, executive function, decision making, working memory, attention, and fatigue.

TABLE III

| | Exemplary Tests and Methods to |
|---|---|
| Cognitive Parameter | Measure Cognitive Parameters |
| Temporal discounting | Kirby Delay Discounting Task |
| Alertness and fatigue | Psychomotor Vigilance Task, go/no-go, |
| | reaction time, |
| Focused attention and | Erikson Flanker Task |
| response inhibition | |
| Working memory | N-Back Task, Change detection, spatial |
| | verbal and visual working memory tests |
| Attentional bias | Dot-Probe Task |
| towards emotional cues | |
| Inflexible persistence | Wisconsin Card Sorting Task |
| Decision making | Iowa Gambling Task |
| Risk taking behavior | Balloon Analogue Risk Task |
| Inhibitory control | Saccade and Anti-Saccade Task |
| Sustained attention | Sustained Attention |
| Executive function | Task Shifting or Set Shifting Task |
| Long term memory | Identifying pictures of famous people |
| | and other memory related tasks |
| Neuropsychological | Montreal Cognitive Assessment (MoCA), Wide |
| test | Range Achievement Test -reading subtest |
| | (WRAT-4), Judgement of Line Orientation |
| | (JOLO), Rey Osterrieth Complex Figure Test |
| | (ROCFT), Hooper Visual Organization Test |
| | (HVOT), Trail Making Test Versions A |
| | and B, Digit Span WAIS4, Phonemic |
| | Fluency FAS, Animal Naming test ANT, |
| | Mini-Mental State Examination (MMSE) |

These cognitive tests can be administered in a clinical/laboratory setting or in a naturalistic, non-clinical setting such as when the user is at home, work or other non-clinical setting. A smart device, such as a smartphone, tablet, or smart watch, can facilitate measuring these cognitive parameters in a naturalistic, non-clinical setting. For example, the Erikson Flanker, N-Back and Psychomotor Vigilance Tasks can be taken via an application on a smart phone, tablet, or smart watch. In one example, the patient can be allowed to explore a virtual reality environment and collect items within the environment. The patient is then asked to recount where each item was found within the virtual environment and the relationship of that location to a starting point, testing the patient's ability to recall spatial relationships among the virtual locations.

TABLE IV provides non-limiting examples of parameters associated with movement and activity of the user, referred to herein alternatively for ease of reference as “motor parameters,” that can be measured and exemplary tests, devices, and methods. The use of portable monitoring, physiological sensing, and portable computing devices allows the motor parameters to be measured. Using embedded accelerometer, GPS, and cameras, the user's movements can be captured and quantified to see how wellness affects them and related to the parameters. Range of motion and gait analysis can be performed in a clinical setting using appropriate motion capture and camera equipment for evaluation.

TABLE IV

| Motor/ | |
|---|---|
| Musculoskeletal | Exemplary Tests and Methods to Measure |
| Parameter | Motor/Musculoskeletal Parameters |
| Activity level | Daily movement total, time of activities, from |
| | wearable accelerometer, steps, Motion Capture data, |
| | gait analysis, GPS, deviation from established |
| | geolocation patterns, force plates |
| Gait analysis | Gait mat, camera, force plates |
| Range of motion | Motion capture, camera, |
| Motor | Time up and go, 6-min walk test, perceptual motor |
| | tasks |

TABLE V provides non-limiting examples of parameters associated with sensory acuity of the user, referred to herein alternatively for ease of reference as “sensory parameters,” that can be measured and exemplary tests, devices, and methods.

TABLE V

| Sensory | Exemplary Tests and Methods to Measure sensory |
|---|---|
| Parameter | Parameters |
| Vision | Visual acuity test, visual field tests, eye tracking, EMG |
| Hearing | Hearing tests |
| Touch | Two-point discrimination, frey filament |
| Smell/taste | |
| Vestibular | Vestibula function test |

TABLE VI provides non-limiting examples of parameters associated with a sleep quantity, phases, and quality of the user, referred to herein alternatively for ease of reference as “sleep parameters,” that can be measured and exemplary tests, devices, and methods.

TABLE VI

| Sleep | Exemplary Tests and Methods to Measure Sleep |
|---|---|
| Parameter | Parameters |
| Sleep from | Sleep onset & offset, sleep quality, sleep quantity, |
| wearables | from wearable accelerometer, temperature, and PPG, |
| Sleep | Pittsburg Sleep Quality Index, Functional Outcomes of |
| Questions | Sleep Questionnaire, Fatigue Severity Scale, Epworth |
| | Sleepiness Scale |
| Devices | Polysomnography; ultrasound, camera, bed sensors, EEG |
| Circadian | Light sensors, actigraphy, serum levels, core body |
| Rhythm | temperature |

TABLE VII provides non-limiting examples of parameters extracted by locating biomarkers associates with the user, referred to herein alternatively for ease of reference as “biomarker parameters,” that can be measured and exemplary tests, devices, and methods. Biomarkers can also include imaging and physiological biomarkers related to a state of chronic wellness and improvement or worsening of the chronic wellness state.

TABLE VII

| | Exemplary Tests and Methods |
|---|---|
| Biomarkers Parameter | to Measure Biomarkers Parameters |
| Genetic biomarkers | Genetic testing |
| Immune biomarkers including | Blood, saliva, and/or urine tests |
| TNF-alpha, immune alteration | |
| (e.g., ILs), oxidative stress, and | |
| hormones (e.g., cortisol) | |

Table VIII provides non-limiting examples of psychosocial and behavioral parameters, referred to herein alternatively for ease of reference as “psychosocial parameters,” that can be measured and exemplary tests, devices, and methods.

TABLE VIII

| Psychosocial or | |
|---|---|
| Behavioral | Exemplary Tests and Methods to Measure |
| Parameter | Psychosocial or Behavioral Parameters |
| Symptom log | Presence of specific symptoms (i.e., fever, |
| | headache, cough, loss of smell) |
| Medical Records | Medical history, prescriptions, setting for treatment |
| | devices such as spinal cord stimulator, imaging data |
| Wellness Rating | Visual Analog Scale, Defense & Veterbrain |
| | wellness rating scale, wellness scale, Wellness |
| | Assessment screening tool and outcomes registry |
| Burnout | Burnout inventory or similar |
| Physical, Mental, | User-Reported Outcomes Measurement Information |
| and Social Health | System (PROMIS), Quality of Life Questionnaire |
| Depression | Hamilton Depression Rating Scale |
| Anxiety | Hamilton Anxiety Rating Scale |
| Mania | Snaith-Hamilton Pleasure Scale |
| Mood/ | Profile of Mood States; Positive Affect Negative |
| Catastrophizing | Affect Schedule |
| scale | |
| Affect | Positive Affect Negative Affect Schedule |
| Impulsivity | Barratt Impulsiveness Scale |
| Adverse Childhood | Childhood trauma |
| Experiences | |
| Daily Activities | Exposure, risk taking |
| Daily Workload | NASA Task Load Index, Perceived Stress Scale |
| and Stress | (PSS), Social Readjustment Rating Scale (SRRS) |
| Social Determents | Social determents of health questionnaire |
| of Health | |

Additional elements of monitoring can include the monitoring of the user's compliance with the use of a smart phone, TV, portable device, a portable device. For example, a user may be sent messages by the system inquiring on their wellness level, general mood, or the status of any other parameter on the portable computing device. A measure of compliance can be determined according to the percentage of these messages to which the user responds via the user interface on the portable computing device and used as an indication as to how likely the patient is to comply with treatment protocols and monitoring. Further, where parameters cannot be readily extracted from wearable or portable devices, they can be retrieved from an electronic health records (EHR) database. Biomarker and motion parameters, in particular, may be retrieved from the EHR, along with other parameters including medical history, prescribed medications, demographic parameters, age, height, weight, and other medically relevant parameters.

At 204, a set of features are determined from the extracted parameters as categorical and continuous values representing the parameters. In one example, the values can include descriptive statistics, such as measures of central tendency (e.g., median, mode, arithmetic mean, or geometric mean) and measures of deviation (e.g., range, interquartile range, variance, standard deviation, etc.) of time series of the monitored parameters, as well as the time series themselves. Specifically, the feature set provided to the predictive model can include, for at least one parameter, either two values representing the value for the parameter at different times or a single value, such as a measure of central tendency or a measure of deviation which represents values for the parameter across a plurality of times.

In one implementation, features can be generated via a wavelet transform on a time series of values for one or more parameters to provide a set of wavelet coefficients. It will be appreciated that the wavelet transform used herein is two-dimensional, such that the coefficients can be envisioned as a two-dimensional array across time and either frequency or scale.

For a given time series of values, xᵢ, the wavelet coefficients, W_{α}(n), produced in a wavelet decomposition can be defined as:

$${W}_{a} ( n ) = {a}^{- 1} {\sum }_{i = 1}^{M} {x}_{i} ψ ( \frac{i - n}{a} ) \qquad Eq . 1$$

wherein ψ is the wavelet function, Mis the length of the time series, and α and n define the coefficient computation locations.

A facial expression classifier (not shown) can evaluate recorded data from a camera and/or recorded images or videos of the user's face from a smartphone or other mobile device, to assign an emotional state to the user at various times throughout the day. The extracted features can be categorical, representing the most likely emotional state of the user, or continuous, for example, as a time series of probability values for various emotional states (e.g., anxiety, discomfort, anger, etc.) as determined by the facial expression classifier. One or more image classifiers that reduce provided medical images to categorical or continuous features for use at the predictive model. It will be appreciated that each of the facial expression classifier and the one or more image classifiers can be implemented using one or more of the models discussed below for use in the predictive model.

In one implementation, the extracted features from the set of parameters can be collected into a set of aggregate parameters. It will be appreciated that each aggregate parameter can be a weighted combination of the set of parameters, functions of parameters from the set of parameters, or features extracted from the parameters. Accordingly, a given aggregate parameter can represent a plurality of parameters, and, in general, the plurality of parameters represented by each aggregate parameter will be related, such that the aggregate parameter represents a specific domain of wellness for the user. In general, each aggregate parameters can use parameters from various sources. In some implementations, the aggregate parameters can be provided to multiple predictive models (not shown) that each receive a unique proper subset of the aggregate parameters. Each predictive model can provide a different clinical parameter representing a different aspect of the user's wellness, such that the aggregate parameters can be utilized for multiple purposes in evaluating the wellness of the user. It will be appreciated that the specific parameters and features used for screening can vary with the implementation.

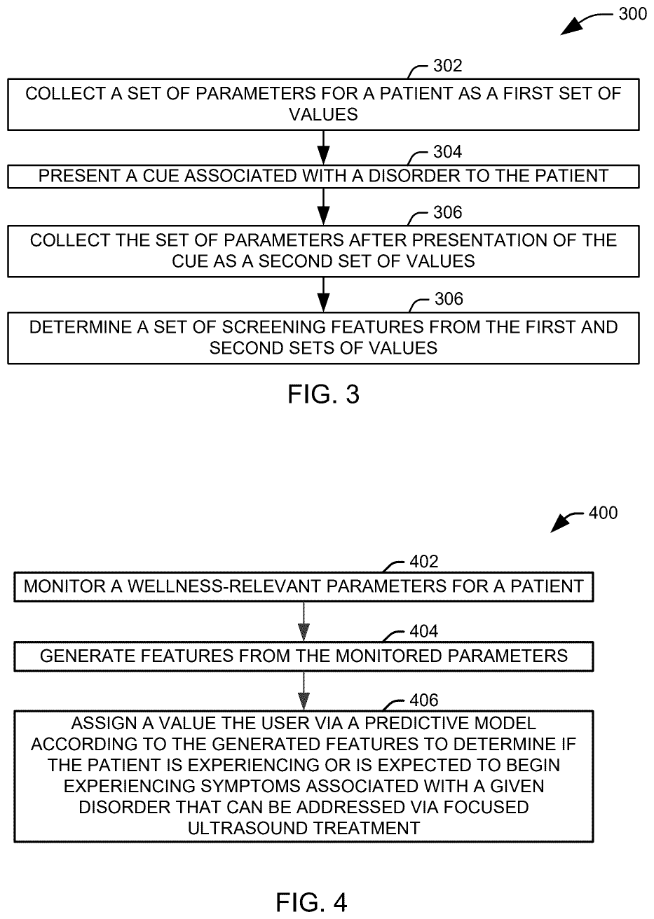

FIG. 3 illustrates a method 300 for using cues to generate features used to screen patients for a disorder. It will be appreciated that the method 300 describes eliciting responses to cues in the context of patient screening, that cue-induced responses can also be used in detecting or predicting the onset of symptoms for a patient, for determining, refining, and optimizing an appropriate target location or appropriate parameters for focused ultrasound treatment, or for evaluating the effectiveness of focused ultrasound treatment. The presentation of cues can also be used as part of treatment for various disorders, as will be discussed in more detail below. At 302, a set of parameters representing the disorder is collected from the patient as a first set of values. The specific parameters will vary with the disorder for which the patient is being screened, but in general, the parameters can include the patient's response to various standard questionnaires as well as physiological parameters measured from the patients. At 304, a cue associated with the disorder is presented to the patient. It will be appreciated that the cue will be selected to be specific to the disorder, and can include not only visual cues but auditory, gustatory, tactile, and olfactory cues. At 306, the set of parameters representing the disorder is collected from the patient after presentation of the cue as a second set of values. At 308, a set of screening features are determined from the first set of values and the second set of values. In one example, the screening features are determined as functions of the first and second values for a given parameter, such as a difference or a ratio between the values.

Returning to FIG. 2, at 206, a clinical parameter is assigned to the patient via a predictive model from the extracted features. The predictive model can utilize one or more pattern recognition algorithms, each of which analyze the extracted features or a subset of the extracted features to assign a continuous or categorical clinical parameter to the user representing the likelihood that the patient would benefit from focused ultrasound treatment. In one example, the clinical parameter can be a continuous parameter representing the likelihood that the patient has a disorder that can be treated via focused ultrasound treatment, the likelihood that the patient has a specific disorder that can be treated via focused ultrasound treatment, the likelihood that a patient will benefit from focused ultrasound treatment generally given a known diagnosis, or the likelihood that the patient will benefit from focused ultrasound treatment in a specific location. In another example, the clinical parameter can be a categorical parameter representing whether the patient has a disorder that can be treated via focused ultrasound treatment, categories representing changes in symptoms associated with a disease or disorder (e.g., “improving”, “stable, “worsening”), categories representing a predicted response to focused ultrasound treatment generally or at a specific location, whether the patient has a specific disorder that can be treated via focused ultrasound treatment, the severity of a disorder, whether a patient will benefit from focused ultrasound treatment given a known diagnosis, or categories representing ranges of likelihoods that the patient falls into one of these categories.

Where multiple classification or regression models are used, an arbitration element can be utilized to provide a coherent result from the plurality of models. The training process of a given classifier will vary with its implementation, but training generally involves a statistical aggregation of training data into one or more parameters associated with the output class. The training process can be accomplished on a remote system and/or on the local device or wearable, app. The training process can be achieved in a federated or non-federated fashion. For rule-based models, such as decision trees, domain knowledge, for example, as provided by one or more human experts or extracted from existing research data, can be used in place of or to supplement training data in selecting rules for classifying a user using the extracted features. Any of a variety of techniques can be utilized for the classification algorithm, including support vector machines, regression models, self-organized maps, fuzzy logic systems, data fusion processes, boosting and bagging methods, rule-based systems, or artificial neural networks.

Federated learning (aka collaborative learning) is a predictive technique that trains an algorithm across multiple decentralized edge devices or servers holding local data samples, without exchanging their data samples. This approach stands in contrast to traditional centralized predictive techniques where all data samples are uploaded to one server, as well as to more classical decentralized approaches which assume that local data samples are identically distributed. Federated learning enables multiple actors to build a common, robust predictive model without sharing data, thus addressing critical issues such as data privacy, data security, data access rights, and access to heterogeneous data. Its applications are spread over a number of industries including defense, telecommunications, IoT, or pharmaceutics.

For example, an SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. In one implementation, the SVM can be implemented via a kernel method using a linear or non-linear kernel.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier. Another example is utilizing an autoencoder to detect outlier in parameters as an anomaly detector to identify when various parameters are outside their normal range for an individual.

Many ANN classifiers are fully connected and feedforward. A convolutional neural network, however, includes convolutional layers in which nodes from a previous layer are only connected to a subset of the nodes in the convolutional layer. Recurrent neural networks are a class of neural networks in which connections between nodes form a directed graph along a temporal sequence. Unlike a feedforward network, recurrent neural networks can incorporate feedback from states caused by earlier inputs, such that an output of the recurrent neural network for a given input can be a function of not only the input but one or more previous inputs. As an example, Long Short-Term Memory (LSTM) networks are a modified version of recurrent neural networks, which makes it easier to remember past data in memory.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used.

In some implementations, the predictive model can be retrained to tune various parameters of the model based upon the accuracy of predictions made by the model. Parameters associated with the model, such as internal weights and thresholds for producing categorical inputs or outputs from continuous values, can be adjusted according to the differences in the actual and predicted outcomes. In one example, an actual value for the clinical parameter for a given patient can be determined as a categorical or continuous outcome, for example, based on a degree of improvement for the patent, a diagnosis, or other appropriate outcome, and combined with the set of features for the patient to provide additional training samples for the model.

If it is determined that focused ultrasound treatment is not appropriate for the patient, the patient can be assigned to an alternative treatment. Alternative treatments can include any of medications, biologicals, surgical intervention, changes of settings for an existing stimulator device, behavioral and social intervention, digital intervention via a portable device, mindfulness approaches, social media approaches, a care provider coming to the individual, directing an individual to go to a clinic, emergency room, or hospital, or directing the user to obtain additional testing.

If it is determined that focused ultrasound treatment is appropriate, a specific focused ultrasound treatment protocol is selected for the patient. It will be appreciated that the specific focused ultrasound treatment protocol can vary with the severity of the disorder. The patient can then monitored to predict or detect an onset of symptoms. It will be appreciated that the timing for focused ultrasound treatment can be personalized and depend on both the onset of symptoms and the progression of the disorder as well as various treatment protocols associated with focused ultrasound modality and any therapeutics introduced during treatment.

FIG. 4 illustrates one method 400 for monitoring a patient to predict or detect an onset of symptoms associated with a disorder for a patient. At 402, parameters are monitored for a patient. It will be appreciated that the monitored parameters can include any of the parameters discussed in Tables II-VIII, and that the parameters can be monitored, for example, using wearable devices, portable devices, such as mobile phones, tablets, and personal computers, or from periodically querying an electronic health records system. At 404, appropriate features are generated from the monitored parameters. At least a subset of the monitored parameters can be represented by features representing a change in the parameter over time, and can be represented as a time series, or a descriptive statistic, such as a measure of central tendency or a measure of deviation which represents values for the parameter across a plurality of times.

At 406, the extracted parameters can be provided to a predictive model to determine if the patient is experiencing or is expected to begin experiences symptoms associated with a given disorder that can be addressed via focused ultrasound treatment. In one example, the predictive model can assign a continuous parameter that corresponds to a likelihood that that user has or is about to have symptoms associated with a disorder, an increase in stress for the patient, an increased in anxiety for the patient, a likelihood that the user will experience an intensifying of symptoms associated with the disorder, a current or predicted level of pain for the user, an expected performance level of the user associated with a current or future time for a particular activity or occupation, or a change in symptoms associated with a disease or disorder. In another example, the predictive model can assign a categorical parameter that corresponds to ranges of the likelihoods described above, the presence or predicted presence of a specific disease or disorder, a set of categories representing the patient's readiness for a particular activity or occupation, categories representing changes in symptoms associated with a disease or disorder (e.g., “improving”, “stable, “worsening”), or categories representing a status of the user (e.g., normal,” “stressed”, “ill”).

In one implementation, the predictive model can include a constituent model that predicts future values for the aggregate parameters, such as a convolutional neural network that is provided with one or more two-dimensional arrays of wavelet transform coefficients as an input. The wavelet coefficients detect changes not only in time, but also in temporal patterns, and can thus reflect changes in the ordinary biological rhythms of the user. It will be appreciated that a given constituent model can use data in addition to the aggregate parameters, such as other extracted features to provide these predictions. Additionally, or alternatively, the predictive model can use constituent models that predict current or future values for the aggregate parameters, with these measures then used as features for generating the output of the predictive model.

In one example, the predictive model is an anomaly detection model, which detects deviations from expected values within a feature space and determines when these deviations are significant. The anomaly detection model can be trained on data from the user, which establishes a baseline of expected values for the user, and/or on data collected from other users. In one example, training the predictive model is initially trained on data collected from other users while values for the subset of the set of parameters are collected from the user over a period of time. Once a sufficient amount of data is available for the user, the predictive model is retrained on the collected values for the subset of the set of parameters.

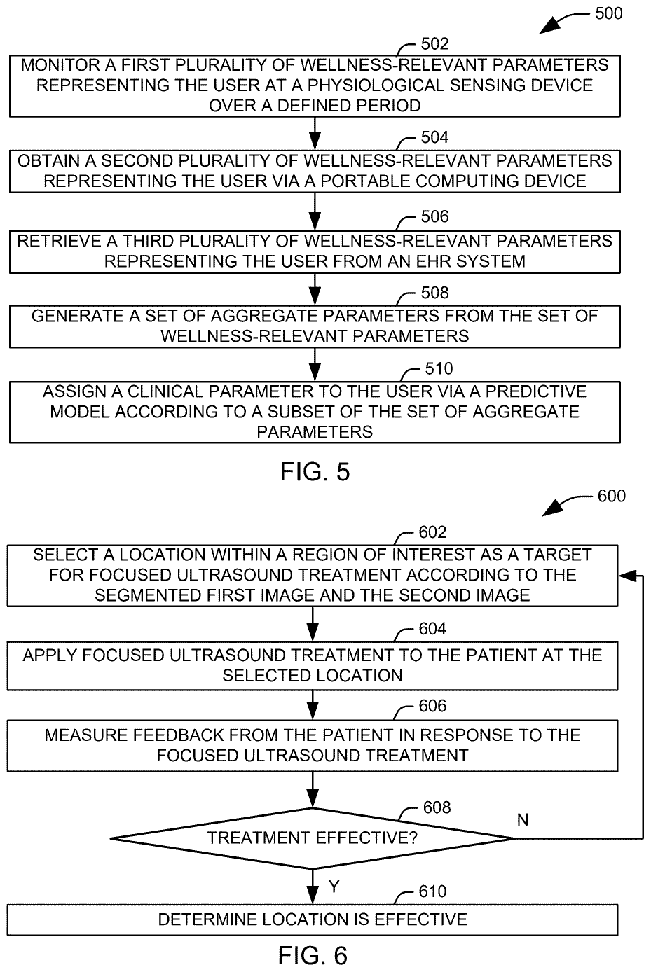

FIG. 5 illustrates another example of a method 500 for evaluating a patient for an onset of symptoms requiring treatment via focused ultrasound treatment. The result of the method is a clinical parameter representing whether the patient is experiencing or is expected to experience symptoms that would require focused ultrasound treatment. At 502, a first plurality of parameters representing the user are monitored at a physiological sensing device over a defined period. In one example, the first plurality of parameters can include parameters representing the autonomic function of the user and parameters representing the sleep and circadian rhythms of the user. At 504, a second plurality of parameters representing the user are obtained via a portable computing device. In one example, the second plurality of parameters can include parameters representing the cognitive and/or socio-behavioral wellness of the user. At 506, a third plurality of parameters representing the user are retrieved from an electronic health records (EHR) system. The third plurality of parameters can include, for example, parameters representing the musculoskeletal health, genomics, and various biomarkers of the user. The first plurality of parameters, the second plurality of parameters, and the third plurality of parameters collectively form a set of parameters.

At 508, a set of aggregate parameters are generated from the set of parameters, with each of the set of aggregate parameters comprising a unique proper subset of the set of parameters. In one example, the set of aggregate parameters includes at least a first aggregate parameter representing autonomic function of the user, a second aggregate parameter representing a cognitive function of the user, and a third aggregate parameter representing a motor and musculoskeletal health of the user. In another example, the set of aggregate parameters includes at least a first aggregate parameter representing sleep and circadian rhythms of the user, a second aggregate parameter representing a socio-behavioral function of the user, and a third aggregate parameter representing a biomarkers and genomics of the user.

At 510, a clinical parameter is assigned to the user via a predictive model according to a subset of the set of aggregate parameters. In one example, the clinical parameter is a value representing an overall wellness of the user, and the subset of the set of aggregate parameters comprises the entire set of aggregate parameters. In another example, the subset of the set of aggregate parameters is a proper subset. It will be appreciated that the aggregate parameters can be provided to multiple predictive models, with each predictive model receiving a unique subset of the set of aggregate parameters. In one example, the predictive model is an anomaly detection model, which detects deviations from expected values within a feature space and determines when these deviations are significant. The anomaly detection model can be trained on data from the user, which establishes a baseline of expected values for the user, or on data collected from other users. In one example, training the predictive model is initially trained on data collected from other users while values for the subset of the set of parameters are collected from the user over a period of time. Once a sufficient amount of data is available for the user, the predictive model is retrained on the collected values for the subset of the set of parameters.

In one example, a wavelet decomposition is performed on the time series for at least one aggregate parameter to provide a set of wavelet coefficients, and the set of wavelet coefficients or one or more values derived from the set of wavelet coefficients can be provided to the predictive model. Additionally or alternatively, the user can be assigned a predicted value representing a future value of a given aggregate parameter according to the values for the subset of aggregate parameters, and the value assigned to the user can be assigned based on the predicted value.

Additionally or alternatively, feedback, in the form of a self-reported level of symptoms, notes from a later clinical visit, or a measured future value for a parameter, can be used to refine the predictive model. For example, the self-reported or measured value can be compared to the value assigned to the user via a predictive model, and a parameter associated with the predictive model can be changed according to the comparison. In one example, this can be accomplished by generating a reward for a reinforcement learning process based on a similarity of the measured outcome to the value assigned to the user and changing the parameter via the reinforcement learning process.

It will be appreciated that each of the parameters and the value assigned to the user can be provided, for example, via a user interface or network interface, to one or more of the user, the user's health care provider, the user's care team, a research team, a user's workplace, a user's sports team, an insurer, or other interested entities. This allows the value to be used to make decisions about the user's care and activities. Feedback provided to the user can be used to improve the user's awareness, perception and interpretation of being in an overall positive and negative states, allowing the user to learn strategies for avoiding negative states and inducing positive states. The provided data can also be used for improvement or optimization of cognitive, motor, sensory, and behavioral function as well as generally attempting to improve the user's quality of life through suggesting actions for the user in response to changes in the clinical parameter. For example, a message can be transmitted to the user's portable computing device suggesting a course of action for the user when the clinical parameter is outside of a predetermined range of values.

FIG. 6 illustrates another method 600 for targeting focused ultrasound treatment in a for treatment of a disorder using acute feedback. At 602, a location within a region of interest is selected as an initial target for focused ultrasound treatment according to the segmented first image and the second image. It will be appreciated that a given treatment can be performed over a number of individual locations, and that this analysis can be performed for each of those locations. At 604, focused ultrasound treatment is applied to the selected location and feedback from the patient in response to the applied focused ultrasound treatment is measured at 606. In one implementation, administration of neuromodulation can be preceded or accompanied by priming, in which that patient is presented with a stimulus associated with their disorder to increase neural activity associated with the disorder. The feedback can include observations of a clinician on the appearance and behavior of the patient, self-reporting from the patient about symptoms of the disorder, measured electrical activity in the region of interest, and biometric parameters, such as those described in Tables II-VIII above. In particular, imaging may be used to determine the effectiveness and potential side effects at a given location. Acoustic feedback can also be used in focused ultrasound applications to evaluate both safety and effectiveness of the treatment. These imaging assessments provide quantitative personalized assessments of therapy safety, dose and benefit, and are analogous to digital fingerprinting.

At 608, an effectiveness of the focused ultrasound treatment is determined according to the measured feedback. This can be done, for example, either by a rule-based approach or by providing the measured feedback parameters to a predictive model. If the modulation is determined to be effective (Y), the location of the focused ultrasound treatment is retained for subsequent focused ultrasound treatments at 610 and the method terminates. If not (N), the selected location is determined to be ineffective, and the method returns to 602 to select a new location within the region of interest as a target for focused ultrasound treatment. The location of the focused ultrasound treatment target is personalized and can be modified live during treatment from acute feedback, or for subsequent treatments. The dosing of the focused ultrasound treatment can also be adjusted.

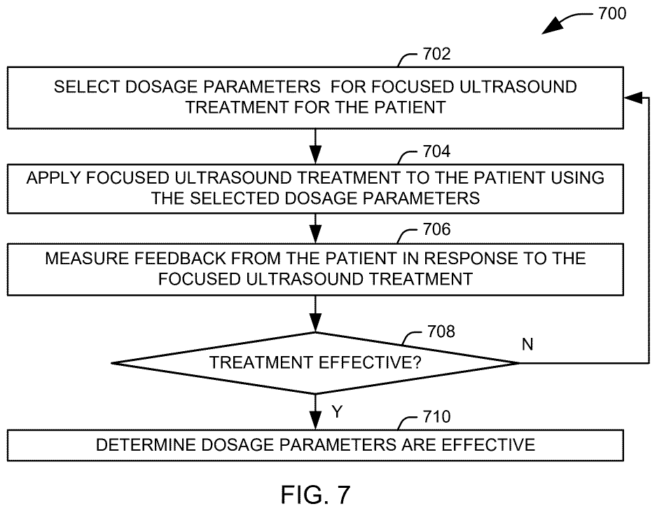

FIG. 7 illustrates a method 700 for selecting a dose for focused ultrasound treatment. It will be appreciated that the dose for focused ultrasound treatment is defined by parameters such as a frequency of application of the focused ultrasound treatment, an energy profile of the focused ultrasound treatment, a shape of the energy field, a direction of the energy field, a pulse rate of the focused ultrasound, a duration of discrete applications of energy, a number of discrete applications of energy, a total duration of the treatment, a type or dosage of a therapeutic provided in concert with the treatment, and an intensity, pressure, or power of the treatment. At 702, an initial dose for the modulation can be selected. It will be appreciated that the initial dose can be standard across patients, but in the illustrated implementation, the initial dose is patient-specific and can be determined according to factors such as a severity of the disorder, a location of the region of interest, and the type of tissue targeted.

At 704, focused ultrasound treatment is applied to the selected location and feedback from the patient in response to the applied focused ultrasound treatment is measured at 706. The feedback can include observations of a clinician on the appearance and behavior of the patient, self-reporting from the patient about symptoms of the disorder, measured electrical activity in the region of interest, and biometric parameters, such as those described in Tables II-VIII above. In particular, imaging may be used to determine the effectiveness and potential side effects of a given treatment. Acoustic feedback can also be used in focused ultrasound applications to evaluate both safety and effectiveness of the treatment.

At 708, an effectiveness of the focused ultrasound treatment is determined according to the measured feedback. This can be done, for example, either by a rule-based approach or by providing the measured feedback parameters to a predictive model. If the modulation is determined to be effective (Y), the selected dosage parameters for the focused ultrasound treatment are retained for subsequent application of energy at 710 and the method terminates. If not (N), the selected dosage parameters are determined to be ineffective, and the method returns to 702 to select new dosage parameters for treatment.

Once the treatment has been applied, feedback is collected from the patient to determine if the treatment has been effective. It will be appreciated that this can be done during or immediately after treatment (“acute feedback”), a short time (e.g., five hours to five days) after a treatment (“subacute feedback”) or a longer time (e.g., more than five days after a treatment (“chronic feedback”). The feedback can be acquired, for example, as any of the parameters listed in Tables II-VIII. To collect acute feedback, the tasks can be presented during or immediately after treatment to determine whether the patient's performance increases, decreases or otherwise changes in response to the treatment. The collected feedback can also include self-reporting from the patient, observations by clinician, measured biometric parameters, such as heart rate variability and blood pressure, and other relevant parameters. In general, the parameters can be adapted based on review by a clinician, a rule-based system that generates suggested changes based on the initial parameters and the measured feedback, or via a predictive model trained on feedback data, treatment parameters, and clinical outcomes for previous patients.

Acute and subacute feedback from presentation application of a treatment can be obtained, for example, by measuring physiological parameters. In some examples, the physiological parameter can be a response of the patient's brain to focused ultrasound treatment and multiple physiological parameters can be measured during any given assessment session. The physiological parameters can be measured via a wearable device such as a ring, watch, or belt or via a smart phone or tablet, for example, in a naturalistic non-clinical setting such as when the patient is at home, work or other non-clinical setting. Exemplary physiological parameters include heart rate, heart rate variability, perspiration, salivation, blood pressure, pupil size, changes in pupil size, eye movements, brain activity, electrodermal activity, body temperature, and blood oxygen saturation level. Table III, above, provides non-limiting examples of physiological parameters that can be measured and exemplary tests to measure the physiological parameters. Sleep parameters can also be measured using wearable devices.

Particularly in treating neurocognitive disorders, cognitive parameters can be assessed by a battery of cognitive tests that measure, for example, executive function, decision making, working memory, attention, and fatigue. Table II, above, provides non-limiting examples of cognitive parameters that are gamified and that can be measured and exemplary methods and tests/tasks to measure such cognitive parameters. These cognitive tests can be administered in a clinical/laboratory setting or in a naturalistic, non-clinical setting such as when the user is at home, work, or other non-clinical setting. A smart device, such as a smartphone, tablet, or smart watch, can facilitate measuring these cognitive parameters in a naturalistic, non-clinical setting. For example, the Erikson Flanker, N-Back and Psychomotor Vigilance Tasks can be taken via an application on a smart phone, tablet, or smart watch.

Behavioral and psychosocial parameters, such as those described in Table VIII above, can measure the user's functionality, such as the user's movement via wearable devices as well as subjective/self-reporting questionnaires. The subjective/self-reporting questionnaires can be collected in a clinical/laboratory setting or in a naturalistic, in the wild, non-clinical setting such as when the user is at home, work, or other non-clinical setting. A smart device, such as a smartphone, tablet, or personal computer can be used to administer the subjective/self-reporting questionnaires. Using embedded accelerometers and cameras, these smart devices can also be used to capture the user's movements as well as facial expression analysis to analyze the user's facial expressions that could indicate mood, anxiety, depression, agitation, and fatigue. A wearable or portable device can also be used to measure parameters representing sleep length, sleep depth, a length of a sleep stage, and heart rate variability.

Treatment provided to the patient is adjusted based upon measured feedback. In general, the treatment can be adapted based on review of the feedback by a clinician, a rule-based system that generates suggested changes based on the initial parameters and the measured feedback, or via a predictive model trained on feedback data, treatment parameters, and clinical outcomes for previous patients. The feedback after focused ultrasound treatment can influence whether further focused ultrasound treatment is provided and how the provided focused ultrasound treatment should be adjusted. In terms of adjusting therapy in the context of focused ultrasound treatment, methods can involve adjusting the parameters or dosing of the focused ultrasound treatment such as, for example, the duration, frequency, or intensity of the focused ultrasound treatment. When the collected data indicates that the patient's condition has not improved, a method can involve adjusting the focused ultrasound treatment so that the focused ultrasound treatment is more effective. For example, if the patient was previously having focused ultrasound (FUS) delivered for five minutes during a therapy session, the patient can have the FUS subsequently delivered for twenty minutes during each session or if the patient was having FUS delivered every thirty days, the patient can have FUS subsequently delivered every two weeks. Conversely, if the parameter measurements indicate improvement, the focused ultrasound treatment parameters may not need adjustment and subsequent focused ultrasound treatment sessions can serve primarily as maintenance sessions or the intensity, frequency or duration of the focused ultrasound treatment can be decreased, for example. The above scenarios are only exemplary and are provided to illustrate that the presence and type of change of the patient's physiological parameter measurement values during and after therapy can influence whether the therapy should be adjusted or terminated.

Further, the degree of the patient's physiological, cognitive, psychosocial, or behavioral parameter measurement value during or after therapy can influence the parameters of subsequent focused ultrasound treatment. For example, if the specific patient seeking therapy has a physiological, cognitive, psychosocial, or behavioral parameter measurement value during or after treatment that is higher than the average parameter measurement value of the same patient population, the therapy can be more aggressive subsequently. Conversely, if the specific patient's parameter measurement value during or after treatment is lower than the average parameter measurement value of the same patient population, the therapy can be less aggressive subsequently. In other words, the severity or degree of the patient's physiological, cognitive, psychosocial, or behavioral parameter measurement value during or after focused ultrasound treatment (as well as baseline values and levels) can correlate to the degree or aggressiveness of future focused ultrasound treatment. The above scenarios are only exemplary and are provided to illustrate that the degree of change of the patient's physiological parameter measurement values during and after focused ultrasound treatment can influence the parameters of subsequent therapy.

In certain aspects, acute, subacute, and chronic feedback each determined from one or more combinations of a physiological, a cognitive, a psychosocial, and a behavioral parameter of the patient after a treatment. For example, obtaining a measurement of baseline values of one or more combinations of a physiological, a cognitive, a psychosocial, and a behavioral parameter of the patient can be obtained. The patient can then be exposed to an initial focused ultrasound signal to a neural target site of the patient. A subsequent measurement can be obtained of resultant values of the one or more combinations of the physiological, the cognitive, the psychosocial, and the behavioral parameter of the patient during or after application of the initial focused ultrasound signal. The resultant values can be compared to the baseline values to determine if the patient's cognitive and/or behavioral functions has improved. The focused ultrasound treatment can be adjusted upon a determination that the patient's cognitive and/or behavioral functions has not improved. For example, if it is determined that the focused ultrasound treatment was not successful, the focused ultrasound treatment can be provided to a different target location.

Form Factor of FUS Delivery and/or Sensors

Devices for transcutaneous FUS neuromodulation can have a variety of configurations. In some instances, a transcutaneous FUS delivery device may be configured as a belt or strap having at least one ultrasound transducer operably attached thereto. A FUS delivery device can be incorporated into clothing (such as, for example, a neck collar, brace, sweater, shirt, pants, socks, glove, stocking, skirt, shoes, underwear, vest, necklace, scarf, wrist band, waist band, ring, other jewelry, sportswear, earpieces, adhesive patches, or stickers. In addition, a FUS device can be embedded in a pillow, a bed, a head rest of a chair, a car seat, a car neck rest, a computer console, and other types of furniture. These devices can provide either ultrasound transducers for delivering a therapy signal, sensors, or a combination of both.

### Addiction

In certain aspects, methods and systems to improve addiction are provided. By improving addiction, the patient's addiction is less severe after FUS therapy than before FUS therapy. For example, the patient's addiction can be improved by reducing the risk factors associated with relapse such as by reducing the patient's substance craving. If craving is reduced, this can contribute to improvements in the patient's mood and anxiety as well as improvements in cognitive aspects such as executive function and impulse control. As such, improving addiction in a patient suffering from addiction includes improving physiological, cognitive, psychosocial, or behavioral characteristics that are the result of or at least partially cause the patient's addiction, such as, for example, anxiety or depression. The patient's addiction cycle can also be improved including use, misuse and addiction. Non-limiting examples of addiction to an addictive behavior include addiction to gambling, food (including binge eating), sex, shopping, sport and physical exercise, video gaming, social media use, pathological working, and compulsive criminal behavior, and combinations thereof. Non-limiting examples of addiction to an addictive chemical substance include addiction to nicotine, alcohol, heroin, opioids, cocaine, benzodiazepines, sedatives/hypnotics, cannabis, amphetamines, other psychoactive substances with abuse liability; and combinations thereof.

Target sites include sites of the limbic system or neural reward circuitry. Non-limiting sites include the nucleus accumbens (including the shell and core of the nucleus accumbens), the anterior cingulate cortex, the posterior cingulate cortex, internal capsule, the insula, the subthalamic nucleus, the striatum including the dorsal and ventral striatum, the prefrontal cortex including the dorsolateral and medial prefrontal cortex, the orbitofrontal cortex, amygdala, or combinations thereof.

### Anxiety Disorders and Anxiety Associated Disorders

As used herein, the term anxiety disorder (which includes anxiety associated disorders) can refer to a dysfunctional state of fear and anxiety, e.g., fear and anxiety that is out of proportion to a stressful situation or the anticipation of a stressful situation. In some instances, an anxiety disorder can be any one or combination of generalized anxiety disorders, such as depression, panic disorder, panic disorder with agoraphobia, agoraphobia, social anxiety disorder, a generalized stress disorder, a stress-induced anxiety disorder, addiction (e.g., alcoholism), an eating disorder and obsessive-compulsive disorder. As used herein, the term eating disorder can refer to any disease or condition that is characterized, at least in part, by obsession with body weight and food as well as abnormal compulsions to avoid eating or uncontrollable impulses to consume abnormally large amounts of food. Eating disorders may affect not only the social well-being, but also the physical well-being of sufferers. Eating disorders can be caused by a symptom of, co-morbid, or otherwise associated with one or more anxiety disorders, mood disorders, neurobehavioral disorders (e.g., psychotic or psychiatric disorders, such as schizophrenia), and/or abnormalities in Body Mass Index, metabolic rate, etc. Non-limiting examples of eating disorders can include anorexia nervosa, bulimia, binge or compulsive eating, obesity, cachexia and wasting syndromes.

As used herein, the term anxiety-associated disorder can refer to a dysfunctional or abnormal psychological and physiological state characterized by cognitive, somatic, emotional, and behavioral components that is related to, caused at least in part by, or correlated with the presence of a co-morbid disease, disorder or condition, such as an eating disorder, autism or other disorder in the autism spectrum. In some instances, these components combine to create the painful feelings typically recognized as anger, fear, apprehension or worry.

It should be appreciated that implementing an ultrasound delivery device as part of a closed-loop system can include placing an ultrasound delivery device on a subject at an autonomic nervous tissue target, sensing a physiological parameter associated with an anxiety or anxiety-associated disorder, and then activating the ultrasound delivery device to apply an ultrasound signal to adjust application of the electrical signal to the neural target site in response to the sensor signal. In some instances, such physiological parameters can include any characteristic, sign, symptom, or function associated with an anxiety or anxiety-associated disorder, such as a chemical moiety or nerve activity (e.g., electrical activity). Examples of such chemical moieties and nerve activities can include the activity of autonomic ganglia (or an autonomic ganglion), the activity of a spinal cord segment or spinal nervous tissue associated therewith, protein concentrations, electrochemical gradients, hormones (e.g., Cortisol), neuroendocrine markers, such as corticosterone, norepinephrine and melatonin, electrolytes, laboratory values, vital signs (e.g., blood pressure), markers of locomotor activity, cardiac markers (e.g., EKG RR intervals), or other signs and biomarkers associated with an anxiety or anxiety-associated disorder.

### Neurodegenerative Disorders

Neurodegenerative disorders can include Alzheimer's disease; mild cognitive impairment (MCI); Lewy body dementia, frontotemporal dementia, vascular dementia, Parkinson's Disease including Parkinson's dementia, Huntington's disease, chronic traumatic encephalopathy, multi-system atrophy, corticobasal degeneration, striatonigral disease, multiple sclerosis, frontotemporal dementia, progressive supranuclear palsy, a condition where the patient has a beta-amyloid protein, a tau protein, and/or another biomarker of a neurodegenerative disorder, and combinations thereof. Such neurodegenerative disorders are exemplary and methods and systems disclosed herein can be used for other neurodegenerative disorders. Methods as disclosed herein can also be used in individuals who have a biomarker of a neurodegenerative disorder but are in a pre-neurodegenerative condition or an early stage of a neurodegenerative disorder, and/or are asymptomatic.

A non-exhaustive list of potential target sites includes the nucleus basalis of meynert, the ventral capsule/ventral striatum, nucleus accumbens, hippocampus, thalamic intralaminar nuclei, other thalamic nuclei, the sub genual cingulate, the fornix, the medial or inferior temporal lobe, the temporal pole, the angular gyrus, the superior or medial frontal lobe, the superior parietal lobe, the precuneus, the supramarginal gyrus, the calcarine sulcus, or combinations thereof.

The neurodegenerative disorder can be improved in a variety of ways. For example, an improvement can comprise a reduction in the concentration of a beta amyloid protein, a tau protein, and/or another biomarker of the neurodegenerative disorder at the target site compared to a baseline/threshold value or a value prior to providing therapy. Other examples of improvements include improvements in cognitive and executive function prior compared to prior to therapy. Other improvements include improvements in visuospatial impairments in Parkinson's disease such as visual perception, blurry vision, judging distances, depth perception, and other visual and spatial deficits that impact so many PD patients. Hallucinations or delusions associated with schizophrenia can also be improved.

### Neuropsychiatric Disorders

A neuropsychiatric disorder can include a dysfunction in neuropsychiatric function. A neuropsychiatric function includes, for example, behavioral expression of brain function such as cognition, initiation, motivation, affect regulation, behavioral control, and perception and/or understanding of emotional stimuli characteristics (including processing speed). Other examples of neuropsychiatric function include general intellectual function, basic attention, complex attention (working memory), executive function, memory (visual and verbal), language, visio-constructional function, and visio-spatial construction. As such, non-limiting examples of neuropsychological functions include general intellectual function; attention, such as basic attention, the ability to monitor and direct attention, and the flexible allocation of attentional resources; working memory or divided attention which refers to a limited-capacity memory system in which information that is the immediate focus of attention can be temporarily held and manipulated (such as, for example, being able to simultaneously maintain two trains of thought in a flexible manner); executive functions which include, for example, planning, problem-solving skills, intentional and self-directed behavior, organizational skills, goal-directed behavior, the ability to generate multiple response alternatives, and maintenance of a conceptual set (i.e. the ability to maintain (or not lose) set or track of what one is doing); the ability to evaluate and modify behavior in response to feedback; verbal and visual memory, or the ability to retain and store new information for future use; visuo-spatial skills, such as judging how lines are oriented or discerning spatial relationships and patterns; visuo-constructional skills including two-dimensional construction skills (such as, for example, drawing or completing puzzles) and three-dimensional constructional skills (such as, for example, arranging blocks to match a design); language such as confrontation naming (such as, for example, naming specific words on demand, such as when shown a picture of the object), word fluency or generating a nonredundant list of words that belong to a specific category; motivation/drive/initiation in the interpersonal, cognitive or behavioral domains; affect regulation (such the ability to control and direct affect and mood in an context appropriate manner); and interpretation of emotion stimuli (such as the ability to interpret emotional facial expressions, posture, body language, prosody, and contextual information in order to infer another's emotional state or help identify an appropriate emotional response). A compromised neuropsychiatric function refers to an abnormality in the neuropsychiatric function compared to a normal, healthy population. Compromised neuropsychiatric function can also include, for example, abnormal anxiety, stress, fear, mood, depression, obsessions, and compulsion. Compromised neuropsychiatric function can be improved in a wide variety of patients who exhibit compromised neuropsychiatric function, including patients suffering from a neuropsychiatric disorder, a psychiatric disorder, a neurodevelopmental disorder, or a neurodegenerative disorder. Non-limiting examples of such disorders include anxiety disorders, addiction, mood disorders, obsessive compulsive disorder (OCD), depression, post-traumatic stress disorder, bipolar disorder, autism, autism spectrum disorder, dyslexia, attention deficit disorder, acquired brain injury, schizophrenia and other forms of dementia, and Parkinson disease. Such conditions are merely examples of conditions where neuropsychological function can be compromised and thus is in need of improvement. As such, methods and systems can be used for conditions where the patient exhibits compromised neuropsychiatric function, such as, for example, cognitive disorders that are inclusive but not limited to mild cognitive impairment (MCI), Lewy body dementia, frontotemporal dementia, vascular dementia, Parkinson's dementia, Chronic traumatic encephalopathy, Huntington's disease, Multi system atrophy, and others. In certain aspects, the neural target site for focused ultrasound can be determined by the presence/concentration of plaques and other biomarkers in the brain.

### Cognitive Disorders

A cognitive disorder ca be a pathological condition in which the patient exhibits compromised cognitive function due to the dysfunction or loss of neurons and/or other nervous system components. Cognitive function includes general intellectual function, basic attention, complex attention (working memory), executive function, memory (visual and verbal), language, visio-constructional function, and visio-spatial construction. As such, non-limiting examples of cognitive functions include general intellectual function; sensory processing, including processing of all sensory input into context; attention, such as basic attention, the ability to monitor and direct attention, and the flexible allocation of attentional resources; working memory or divided attention which refers to a limited-capacity memory system in which information that is the immediate focus of attention can be temporarily held and manipulated (such as, for example, being able to simultaneously maintain two trains of thought in a flexible manner); executive functions which include, for example, planning, problem-solving skills, intentional and self-directed behavior, organizational skills, goal-directed behavior, the ability to generate multiple response alternatives, and maintenance of a conceptual set (i.e. the ability to maintain (or not lose) set or track of what one is doing); the ability to evaluate and modify behavior in response to feedback; verbal and visual memory, or the ability to retain and store new information for future use; visuo-spatial skills, such as judging how lines are oriented or discerning spatial relationships and patterns; visuospatial function; higher order processing of visual input; visuo-constructional skills including two-dimensional construction skills (such as, for example, drawing or completing puzzles) and three-dimensional constructional skills (such as, for example, arranging blocks to match a design); language such as confrontation naming (such as, for example, naming specific words on demand, such as when shown a picture of the object), word fluency or generating a nonredundant list of words that belong to a specific category; and combinations thereof.

Examples include Alzheimer's disease and related dementias, developmental disorders, such as autism, and other cognitive disorders such as schizophrenia, mild cognitive impairment (MCI), Lewy body dementia, frontotemporal dementia, vascular dementia, Parkinson's dementia, Chronic traumatic encephalopathy, Huntington's disease, and multi system atrophy, corticobasal degeneration, striatonigral disease, multiple sclerosis, conditions where the patient has a beta-amyloid protein, a tau protein, and/or another biomarker of a neurodegenerative disorder, and combinations thereof.

Non-limiting examples of target sites include the hippocampus, nucleus accumbens, insula, cingulate cortex (anterior and/or) posterior, DLPFC, subthalamic nucleus, globus palldus, a thalamic nucleus, and combinations thereof.

### Neurodevelopmental Disorders

As used herein, the term autism can refer to a disease or disorder characterized by impaired social interaction and communication, and by restricted and repetitive behavior. In some instances, autism can refer to one of three recognized disorders in the autism spectrum, the other two being Asperger syndrome and pervasive developmental disorder. Non-limiting examples of target sites to treat the cognitive and behavioral aspects of neurodevelopmental disorders can include the nucleus accumbens, the ventral striatum, the ventral internal capsule, and thalamic nuclei, the pallidum, or combinations thereof.

### Post-Traumatic Stress Disorder

A patient diagnosed with PTSD can refer to having a diagnosis of at least one sign, symptom, or symptom cluster indicative of PTSD. Non-limiting examples of such traumatic events can include military combat, terrorist incidents, physical assault, sexual assault, motor vehicle accidents, and natural disasters.

The Diagnostic and Statistical Manual of Mental Disorders-IV-Text revised (DSM-IV-TR), a handbook for mental health professionals that lists categories of mental disorders and the criteria, classifies PTSD as an anxiety disorder. According to the DSM-IV-TR, a PTSD diagnosis can be made if:
1. the patient experienced, witnessed, or was confronted with an event or events that involved actual or threatened death or serious injury, or a threat to the physical integrity of self or others and the response involved intense fear, helplessness, or horror;
2. as a consequence of the traumatic event, the patient experiences at least one re-experiencing/intrusion symptom, three avoidance/numbing symptoms, and two hyperarousal symptoms, and the duration of the symptoms is for more than 1 month; and
3. the symptoms cause clinically significant distress or impairment in social, occupational, or other important areas of functioning.

In some instances, if the patient's disorder fulfills DSM-IV-TR criteria, the patient is diagnosed with PTSD. In other instances, if the patient has at least one sign, symptom, or symptom cluster of PTSD, the patient is diagnosed with PTSD. In further instances, a scale can be used to measure a sign, symptom, or symptom cluster of PTSD, and PTSD can be diagnosed on the basis of the measurement using that scale. In some instances, a “score” on a scale can be used to diagnose or assess a sign, symptom, or symptom cluster of PTSD. In other instances, a “score” can measure at least one of the frequency, intensity, or severity of a sign, symptom, or symptom cluster of PTSD.

The term scale can refer to a method to measure at least one sign, symptom, or symptom cluster of PTSD in a patient. In some instances, a scale may be an interview or a questionnaire. Non-limiting examples of scales include Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IBS), Impact of Event Scale-Revised (IES-R), Clinical Global Impression Scale (CGI), Clmical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Asb erg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI).

The terms sign and signs can refer to objective findings of a disorder (e.g., PTSD). In some instances, a sign can be a physiological manifestation or reaction of a disorder (e.g., PTSD). For example, a sign may include heart rate and rhythm, body temperature, pattern and rate of respiration, papillary changes and blood pressure. In other instances, signs can be associated with, or indicative of, symptoms.

The terms symptom and symptoms can refer to subjective indications that characterize a disorder. Symptoms of PTSD may refer to, for example, recurrent and intrusive trauma recollections, recurrent and distressing dreams of the traumatic event, acting or feeling as if the traumatic event were recurring, distress when exposed to trauma reminders, physiological reactivity when exposed to trauma reminders, efforts to avoid thoughts or feelings associated with the trauma, efforts to avoid activities or situations, inability to recall trauma or trauma aspects, markedly diminished interest in significant activities, feelings of detachment or estrangement from others, restricted range of affect, sense of a foreshortened future, social anxiety, anxiety with unfamiliar surroundings, difficulty falling or staying asleep, irritability or outbursts of anger, difficulty concentrating, hypervigilance, and exaggerated startle response. In some instances, the physiological reactivity manifests in at least one of abnormal respiration, abnormal cardiac rate of rhythm, abnormal blood pressure, abnormal function of a special sense, and abnormal function of sensory organ. In other instances, restricted range of effect characterized by diminished or restricted range or intensity of feelings or display of feelings can occur and a sense of a foreshortened future can manifest in thinking that one will not have a career, marriage, children, or a normal life span. In further instances, children and adolescents may have symptoms of PTSD, such as disorganized or agitated behavior, repetitive play that expresses aspects of the trauma, frightening dreams which lack recognizable content, and trauma-specific reenactment.

The term symptom cluster can refer to a set of signs, symptoms, or a set of signs and symptoms that are grouped together because of their relationship to each other or their simultaneous occurrence. In some instances, for example, PTSD is characterized by three symptom clusters: re-experiencing/intrusion; avoidance/numbing; and hyperarousal.

The term re-experiencing/intrusion can refer to at least one of recurrent and intrusive trauma recollections, recurrent and distressing dreams of the traumatic event, acting or feeling as if the traumatic event were recurring, distress when exposed to trauma reminders, and physiological reactivity when exposed to trauma reminders. In some instances, the physiological reactivity can manifest in at least one of abnormal respiration, abnormal cardiac rate of rhythm, abnormal blood pressure, abnormal function of a special sense, and abnormal function of sensory organ.

The term avoidance/numbing can refer to at least one of efforts to avoid thoughts or feelings associated with the trauma, efforts to avoid activities or situations, inability to recall trauma or trauma aspects, markedly diminished interest in significant activities, feelings of detachment or estrangement from others, restricted range of affect, and sense of a foreshortened future. Restricted range of effect characterized by diminished or restricted range or intensity of feelings or display of feelings can occur. A sense of a foreshortened future can manifest in thinking that one will not have a career, marriage, children, or a normal life span. Avoidance/numbing can also manifest in social anxiety and anxiety with unfamiliar surroundings.

The term hyperarousal can refer to at least one of difficulty falling or staying asleep, irritability or outbursts of anger, difficulty concentrating, hypervigilance, and exaggerated startle response.

Non-limiting examples of target sites include the nucleus accumbens (including the shell and core of the nucleus accumbens), the anterior cingulate cortex, the posterior cingulate cortex, the internal capsule, the insula, the subthalamic nucleus, the striatum including the dorsal and ventral striatum, the prefrontal cortex including the dorsolateral and medial prefrontal cortex, the orbitofrontal cortex, the amygdala, the extended amygdala, or combinations thereof.

The disclosed systems and methods can be used to initially treat a patient, screen or select a patient for therapy, and adjust follow up ultrasound therapy sessions. Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Further, unless otherwise specified, none of the steps of the methods of the present disclosure are confined to any particular order of performance. Furthermore, all references cited herein are incorporated by reference in their entirety.

What is claimed is:

1. A method of improving a medical condition in a patient in need thereof comprising:
delivering a focused ultrasound neuromodulation signal to a neural target site of the patient's brain, the ultrasound signal having a mechanical index of between about 1.0 and 8.0 and/or an acoustic pressure of between about 1.0 MPa to about 3.0 MPa, the medical condition comprising addiction, binge eating, tinnitus, anxiety disorders and anxiety associated disorders, neurodegenerative disorders, neuropsychiatric disorders, cognitive disorders, neurodevelopmental disorders, post-traumatic stress disorder, stroke recovery, and combinations thereof; and
improving the patient's medical condition.

2. The method of claim 1, further comprising:
screening a patient to determine if the focused ultrasound neuromodulation signal is appropriate for the patient to treat the medical condition;
monitoring the patient to measure a plurality of parameters for the patient and detect or predict an onset of symptoms associated with the disorder from the plurality of parameters if the focused ultrasound neuromodulation signal has been determined to be appropriate for the patient;
selecting a location within the neural target site according to at least one of the parameters when an onset of symptoms has been detected or predicted;
selecting a dosage parameter associated with the focused ultrasound neuromodulation signal according to at least one of the plurality of parameters, the focused ultrasound neuromodulation signal being delivered with the selected dosage parameter; and
measuring the plurality of parameters after the focused ultrasound neuromodulation signal is delivered to determine an effectiveness of the focused ultrasound neuromodulation signal.

3. The method of claim 2, wherein the location is a first location and the method further comprises:
determining, from the plurality of parameters, that the focused ultrasound neuromodulation signal is not effective in treating the medical condition;
selecting a second location within the neural target site; and
delivering the focused ultrasound neuromodulation signal to the second location.

4. The method of claim 2, wherein the dosage parameter is a first dosage parameter and the method further comprises:
determining, from the plurality of parameters, that the focused ultrasound neuromodulation signal is not effective in treating the medical condition;
selecting a second dosage parameter according to at least one of the plurality of parameters; and
delivering the focused ultrasound neuromodulation signal using the second dosage parameter.

5. The method of claim 2, wherein the method further comprises:
determining, from the plurality of parameters, that the focused ultrasound neuromodulation signal is effective in treating the medical condition;
predicting an onset of symptoms for the patient from the plurality of parameters; and
delivering the focused ultrasound neuromodulation signal to the location within the neural target site in response to predicting the onset of symptoms.

6. The method of claim 2, wherein the method further comprises:
determining, from the plurality of parameters, that the focused ultrasound neuromodulation signal is not effective in treating the medical condition; and
selecting an alternative therapy for the medical condition.

7. A non-transitory computer accessible medium having stored thereon computer executable instructions which when executed by a processor causes the steps of:
controlling an ultrasound pulse generator to deliver a focused ultrasound neuromodulation signal to a neural site of the brain, the ultrasound signal having a mechanical index of between about 0.1 to about 8.0 and/or an acoustic pressure of between about 0.1 MPa to about 3.0 MPa and being sufficient to improve chronic or refractory rhinitis, chronic rhinosinusitis, autonomic instability/autonomic dysfunction, functional gastrointestinal disorders, inflammatory disorders, immune disorders, complex regional pain syndrome, post-traumatic stress disorder (PTSD), anxiety and anxiety-associated disorders, autism, fibromyalgia, uterine function, metabolic disorder, urinary incontinence, or binge eating.
